(19) United States
(12) Patent Application Publication
(10) Pub. No.: US 2022/0160208 A1
(43) Pub. Date: May 26, 2022

(54) Methods and Systems for Cystoscopic Imaging Incorporating Machine Learning

(71) Applicants: The Board of Trustees of the Leland Stanford Junior University, Stanford, CA (US); U.S. Government Represented by the Department of Veterans Affairs, Washington, DC (US)

(72) Inventors: Joseph C. Liao, Stanford, CA (US); Lei Xing, Stanford, CA (US); Eugene Shkolyar, Stanford, CA (US); Xiao Jia, Stanford, CA (US)

(73) Assignees: The Board of Trustees of the Leland Stanford Junior University, Stanford, CA (US); U.S. Government Represented by the Department of Veterans Affairs, Washington, DC (US)

(21) Appl. No.: 17/601,377

(22) PCT Filed: Apr. 3, 2020
(86) PCT No.: PCT/US20/26697

§ 371 Date: Oct. 4, 2021

Related U.S. Application Data
(60) Provisional application No. 62/828,924, filed on Apr. 3, 2019.

(51) Int. Cl. A61B 1/00; A61B 1/307; A61B 1/06; G06T 7/10

(52) U.S. Cl. CPC A61B 1/000096; A61B 1/307; A61B 1/06; G06T 7/10; G06T 2207/10016; G06T 2207/30096; G06T 2207/20081; G06T 2207/20084; G06T 2207/10068

(57) ABSTRACT

Over 2 million cystoscopies are performed annually in the United States and Europe for detection and surveillance of bladder cancer. Adequate identification of suspicious lesions is critical to minimizing recurrence and progression rates, however standard cystoscopy misses up to 20% of bladder cancer. Access to adjunct imaging technology may be limited by cost and availability of experienced personnel. Machine learning holds the potential to enhance medical decision-making in cancer detection and imaging. Various embodiments described herein are directed to methods for identifying cancers, tumors, and/or other abnormalities present in a person's bladder. Additional embodiments are directed to machine learning systems to identify cancers, tumors, and/or other abnormalities present in a person's bladder, while additional embodiments will also identify benign or native structures or features in a person's bladder. Further embodiments incorporate such systems into cystoscopy equipment to allow for real time and/or immediate detection of cancers, tumors, and/or other abnormalities present in a person's bladder during a cystoscopy procedure.

### CROSS REFERENCE TO RELATED APPLICATIONS

This application claims priority to U.S. Provisional Application Ser. No. 62/828,924, entitled “Methods and Systems for Cystoscopic Imaging Incorporating Machine Learning” to Liao et al., filed April 3, 2019; the disclosure of which is herein incorporated by reference in its entirety.

### FIELD OF THE DISCLOSURE

The present disclosure relates to cystoscopic imaging, specifically, methods and systems incorporating machine learning algorithms for detecting cancers, tumors, and other abnormalities.

### BACKGROUND OF THE DISCLOSURE

Bladder cancer (BCa) is the sixth most common malignancy in the United States, with an estimated 81,190 new diagnoses in 2018. (See Society AC. Cancer Facts and Figures 2018. Atlanta Am. Cancer Soc. 2018; the disclosure of which is hereby incorporated by reference in its entirety.) Hematuria is the most common symptom leading to BCa screening, the prevalence of which is as high as 18% in the general population. (See Freni SC, Freni-Titulaer L W. Microhematuria found by mass screening of apparently healthy males. Acta Cytol; 21: 421-23 and Mohr D N, Offord K P, Owen R A, Melton L J. Asymptomatic microhematuria and urologic disease. A population-based study. JAMA 1986; 256: 224-29; the disclosures of which are hereby incorporated by reference in their entirety.) Non-muscle invasive bladder cancer (NMIBC), which is typically managed endoscopically, accounts for 75% of new BCa diagnoses. High recurrence and progression rates necessitate frequent surveillance and intervention, thus making BCa one of the most expensive cancer to treat in the U.S per lifetime. (See Chamie K, Litwin M, Bassett J, Daskivich T. Recurrence of high risk bladder cancer: A population based analysis. Cancer 2013; 70: 646-56; Park J C, Hahn N M. Bladder cancer: A disease ripe for major advances. Clin Adv Hematol Oncol 2014; 12: 838-45; atek RS, Hollenbeck B K, Holmang S, et al. The Economics of Bladder Cancer: Costs and Considerations of Caring for This Disease. Eur Urol 2014; 66: 253-62; and Yeung C, Dinh T, Lee J. The Health Economics of Bladder Cancer: An Updated Review of the Published Literature. Pharmacoeconomics 2014; 32: 1093-104; the disclosures of which are hereby incorporated by reference in their entirety.) The standard for diagnosis and surveillance of bladder cancer is outpatient white light cystoscopy (WLC), and it is estimated that over two million cystoscopies are performed in the United States and European Union annually. (See Yeung C, Dinh T, Lee J. The Health Economics of Bladder Cancer: An Updated Review of the Published Literature. Pharmacoeconomics 2014; 32: 1093-104 and Langston JP, Duszak R, Orcutt VL, et al. The Expanding Role of Advanced Practice Providers in Urologic Procedural Care. Urology 2017; 106: 70-75; the disclosures of which are hereby incorporated by reference in their entirety.) Suspicious findings on cystoscopy prompt transurethral resection of bladder tumors (TURBT) for histopathologic examination and treatment.

Adequate identification and complete resection of NMIBC reduces recurrence and progression rates. (See Hermann G G, Mogensen K, Carlsson S, Marcussen N, Duun S. Fluorescence-guided transurethral resection of bladder tumours reduces bladder tumour recurrence due to less residual tumour tissue in Ta/T1 patients: A randomized two-centre study. BJU Int 2011. DOI:10.1111/j.1464-410X.2011.10090.x; and Alfred Witjes J, Palou Redorta J, Jacqmin D, et al. Hexaminolevulinate-Guided Fluorescence Cystoscopy in the Diagnosis and Follow-Up of Patients with Non-Muscle-Invasive Bladder Cancer: Review of the Evidence and Recommendations. Eur Urol; 57: 607-14; the disclosures of which are hereby incorporated by reference in their entirety.) Despite this, up to 40% of patients presenting with multifocal disease have an incomplete initial resection. (See Alfred Witjes J, Palou Redorta J, Jacqmin D, et al. Hexaminolevulinate-Guided Fluorescence Cystoscopy in the Diagnosis and Follow-Up of Patients with Non-Muscle-Invasive Bladder Cancer: Review of the Evidence and Recommendations. Eur Urol; 57: 607-14; Burger M, Grossman H B, Droller M, et al. Photodynamic diagnosis of non-muscle-invasive bladder cancer with hexaminolevulinate cystoscopy: A meta-analysis of detection and recurrence based on raw data. Eur Urol 2013. DOI:10.1016/j.eururo.2013.03.059; and Brausi M, Collette L, Kurth K, et al. Variability in the Recurrence Rate at First Follow-up Cystoscopy after TUR in Stage Ta T1 Transitional Cell Carcinoma of the Bladder: A Combined Analysis of Seven EORTC Studies. Eur Urol 2002; 41: 523-31; the disclosures of which are hereby incorporated by reference in their entirety.) Standard WLC misses up to 15% of papillary bladder tumors and 30% of flat lesions. (See Grossman HB, Gomella L, Fradet Y, et al. A Phase III, Multicenter Comparison of Hexaminolevulinate Fluorescence Cystoscopy and White Light Cystoscopy for the Detection of Superficial Papillary Lesions in Patients With Bladder Cancer. J Urol 2007; 178: 62-67 and Daneshmand S, Bazargani S T, Bivalacqua TJ, et al. Blue light cystoscopy for the diagnosis of bladder cancer: Results from the US prospective multicenter registry. Urol Oncol Semin Orig Investig 2018: 1-6; the disclosures of which are hereby incorporated by reference in their entirety.) Given the high rate of missed bladder tumors on WLC adjunct imaging technologies have been introduced to improve detection. Photodynamic diagnosis (PDD) is the most widespread enhanced cystoscopy technique. PDD requires the instillation of a photosensitizer that is absorbed by the urothelium and accumulates preferentially in neoplastic cells. The photosensitizer can then be seen under blue light. Although effective at detecting additional tumors and reducing recurrence, PDD is limited by the need to instill an intravesical contrast agent, reliance on specialized equipment, high false-positive rate, and learning curve. (See Daneshmand S, Patel S, Lotan Y, et al. Efficacy and Safety of Blue Light Flexible Cystoscopy with Hexaminolevulinate in the Surveillance of Bladder Cancer: A Phase III, Comparative, Multicenter Study. J Urol 2018; 199: 1158-65; the disclosure of which is hereby incorporated by reference in its entirety.) Other optical imaging techniques have been developed to aid in bladder cancer diagnosis but integration into clinical practice has been limited. (See Sonn GA, Jones SNE, Tarin T V., et al. Optical Biopsy of Human Bladder Neoplasia With In Vivo Confocal Laser Endomicroscopy. J Urol 2009; 182: 1299-305; Kim S Bin, Yoon SG, Tae J, et al. Detection and recurrence rate of transurethral resection of bladder tumors by narrow-band imaging: Prospective, randomized comparison with white light cystoscopy. Investig Clin Urol 2018; 59: 98; and Lerner SP, Goh A C, Tresser N J, Shen S S. Optical Coherence Tomography as an Adjunct to White Light Cystoscopy for Intravesical Real-Time Imaging and Staging of Bladder Cancer. Urology 2008. DOI:10.1016/j.urology.2008.02.002; the disclosures of which are hereby incorporated by reference in their entirety.)

In addition to the challenges related to performing high-quality cystoscopy, the high volume of cystoscopies performed annually represents a public health challenge. The urologic work force is shrinking in the face of rising demand from an aging population, impacting access and availability. (See McKibben M J, Kirby E W, Langston J, et al. Projecting the Urology Workforce Over the Next 20 Years. Urology 2016; 98: 21-26; the disclosure of which is hereby incorporated by reference in its entirety.) As a result, long wait-times for standard procedures are impacting health-care systems. This has prompted efforts internationally to train advanced practitioners and non-urologists to perform WLC, however there has been limited adaptation of this practice. Discrepancies in performance between trainees and experienced urologists likely contribute to the under-utilization of non-urologists for standard WLC. (See MacKenzie K R, Aning J. Defining competency in flexible cystoscopy: a novel approach using cumulative Sum analysis. BMC Urol 2016; 16: 31; the disclosure of which is hereby incorporated by reference in its entirety.) Likewise, demonstrable differences in performance of TURBT can be seen between novice and seasoned practitioners. (See Bos D, Allard C B, Dason S, Ruzhynsky V, Kapoor A, Shayegan B. Impact of resident involvement in endoscopic bladder cancer surgery on pathological outcomes. Scand J Urol 2016; 50: 234-38; the disclosure of which is hereby incorporated by reference in its entirety.) The lack of a standardized cystoscopy reporting system makes communication between providers challenging, and may result in repeat procedures to confirm previous findings. In the surveillance setting, inter- and intra-provider variability in documentation makes interpretation of changes in the bladder on serial evaluation challenging.

Over 2 million cystoscopies are performed annually in the United States and Europe for detection and surveillance of bladder cancer. Adequate identification of suspicious lesions is critical to minimizing recurrence and progression rates, however standard cystoscopy misses up to 20% of bladder cancer. Access to adjunct imaging technology may be limited by cost and availability of experienced personnel. Machine learning holds the potential to enhance medical decision-making in cancer detection and imaging.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature.

In one embodiment, a method for identifying a bladder tumor includes obtaining a video of a cystoscopic exam, segmenting an area of concern present in the video, recording details about the area of concern, and providing details about the area of concern to a practitioner.

In a further embodiment, the obtaining step is obtained from a live cystoscopic exam.

In another embodiment, the live cystoscopic exam is accomplished using white light cystoscopy.

In a still further embodiment, the segmenting an area of concern step uses a machine learning algorithm comprising a convolutional neural network.

In still another embodiment, the convolutional neural network is trained with annotated cystoscopic video.

In a yet further embodiment, the annotated cystoscopic video includes annotations of abnormal tissues and benign physiologies.

In yet another embodiment, the convolutional neural network comprises two stages.

In a further embodiment again, the convolutional neural network comprises a first stage and a second stage, wherein the first stage highlights an area of concern and the second stage segments a tumor.

In another embodiment again, the providing step is accomplished via video overlay during a subsequent cystoscopic exam.

In a further additional embodiment, the method further includes obtaining patient information, wherein the patient information comprises at least one of the group consisting of: age, sex, gender, and medical history.

In another additional embodiment, the segmenting step highlights the area of concern on a video monitor.

In a still yet further embodiment, the method further includes characterizing the area of concern.

In still yet another embodiment, the characterizing step comprises at least one of the group consisting of: identifying the area of concern, locating the area of concern, and determining the size of the area of concern.

In a still further embodiment again, the characterizing step comprises identifying the area of concern and excluding the area of concern, if the area of concern is benign.

In still another embodiment again, the method further includes treating the patient for a tumor.

In a still further additional embodiment, where treating the patient comprises at least one of the group consisting of: resecting the tumor, introducing an anti-cancer drug to the bladder, and introducing an anti-cancer drug to the tumor.

In still another additional embodiment, a method for treating a bladder tumor includes obtaining a video from a live white light cystoscopic exam, obtaining patient information, wherein the patient information comprises at least one of the group consisting of: age, sex, gender, and medical history, segmenting an area of concern present in the video using a machine learning algorithm including a convolutional neural network trained with annotated cystoscopic video, where the annotated video includes annotations of abnormal tissues and benign physiologies, where the segmenting step highlights the area of concern on a video monitor, characterizing the area of concern, where characterizing the area of concern includes at least one of the group consisting of: identifying the area of concern, locating the area of concern, and determining the size of the area of concern, where characterizing the area of concern further includes excluding the area of concern, if the area of concern is benign, recording details about the area of concern, providing details about the area of concern to a practitioner, and treating the patient for a tumor; where treating the patient includes at least one of the group consisting of: resecting the tumor, introducing an anti-cancer drug to the bladder, and introducing an anti-cancer drug to the tumor.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates a schematic of a machine learning algorithm in accordance with various embodiments of the invention.

FIGS. 2A-2M illustrate segmentation and highlighting of abnormal tissues in accordance with various embodiments of the invention.

FIGS. 3A-3D illustrate annotated data in accordance with various embodiments of the invention.

FIG. 4 illustrates a method in accordance with various embodiments of the invention.

FIGS. 5A-5C illustrate an exclusionary process of benign features in accordance with various embodiments of the invention.

FIG. 6A illustrates the identification of a flat tumor in accordance with various embodiments of the invention.

FIG. 6B illustrates confirmation of a flat tumor using blue light cystoscopy in accordance with various embodiments of the invention.

FIG. 7 illustrates an ROC curve determined on a training set across a range of thresholds in accordance with various embodiments.

### DETAILED DESCRIPTION OF THE DISCLOSURE

The evolution of machine learning over recent years has allowed for automation in the field of cancer imaging. (See Zhong X, Cao R, Shakeri S, et al. Deep transfer learning-based prostate cancer classification using 3 Tesla multi-parametric MRI. Abdom Radiol 2018; published online Nov 20. DOI:10.1007/s00261-018-1824-5 and Coy H, Hsieh K, Wu W, et al. Deep learning and radiomics: the utility of Google TensorFlowTM Inception in classifying clear cell renal cell carcinoma and oncocytoma on multiphasic CT. Abdom Radiol 2019; published online Feb 18. DOI:10.1007/s00261-019-01929-0; the disclosures of which are hereby incorporated by reference in their entirety.) When applied to endoscopy, deep-learning has been shown to detect polyps on colonoscopy with excellent sensitivity and specificity, and early work on classifying images from a cystoscopy atlas has been promising. (See Wang P, Xiao X, Glissen Brown J R, et al. Development and validation of a deep-learning algorithm for the detection of polyps during colonoscopy. Nat Biomed Eng 2018; 2: 741-48; Eminaga O, Eminaga N, Semjonow A, Breil B. Diagnostic Classification of Cystoscopic Images Using Deep Convolutional Neural Networks. 2018; : 1-8; and Gosnell ME, Ph D, Polikarpov DM, et al. Computer-assisted cystoscopy diagnosis of bladder cancer. Urol Oncol Semin Orig Investig 2018; 36: 8.e9-8.e15; the disclosures of which are hereby incorporated by reference in their entirety.) Despite the high prevalence of BCa, there have been no large-scale investigations of deep-learning for bladder tumor detection on cystoscopy. Machine learning holds the potential to enhance medical decision-making in BCa detection and imaging.

Many embodiments described herein utilize one or more machine learning algorithms for augmented detection of bladder cancer during standard cystoscopy. Many embodiments incorporate machine learning algorithms into a cystoscopic system to detect bladder cancers, bladder tumors, inflammation, and/or any other physiology within a bladder. Numerous embodiments are platform agnostic, such that the machine learning algorithm can be combined with any cystoscopic system, including white light cystoscopes, blue light cystoscopes, or any other system of cystoscopy.

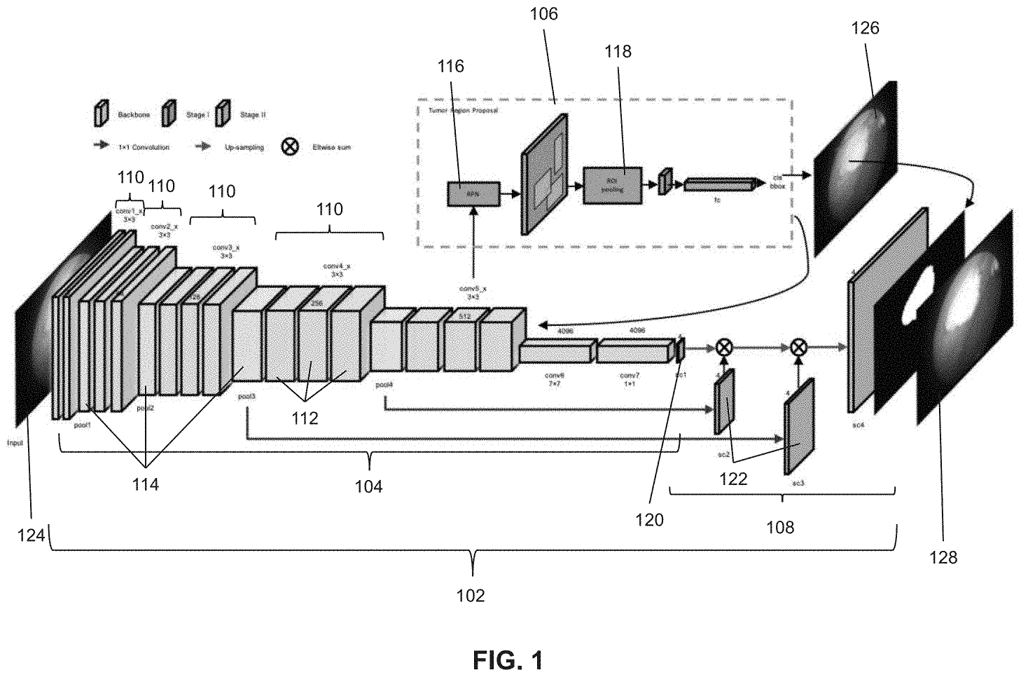

Additional embodiments will use multiple algorithms to accomplish tumor identification and segmentation. Some of these multi-algorithm embodiments use interrelated data, such that the information from one is directly used by the other and/or both algorithms use the same input data. In some of such embodiments, the first algorithm can be used to identify abnormal tissue and highlight the region of the abnormal tissue, while the second algorithm can segment the tumor. Certain embodiments will run the algorithms simultaneously, such that the segmentation can be provided in real time, while additional embodiments can run the algorithms sequentially. An example of a multi-algorithm embodiment is illustrated in FIG. 1, illustrating a convolutional neural network 102. Many embodiments of the convolutional neural network 102 comprising a backbone 104. Additional embodiments further comprise a first stage 106 to highlight regions of interest and/or a second stage 108 to segment images. In many embodiments backbone 104 comprises a series of convolutional blocks 110. Each convolutional block 110 comprises a number of convolutions 112 (e.g., 1-5 convolutions 112 per convolutional block 110). Many embodiments possess pooling layers 114 between certain convolutional blocks 110.

The first stage 106 of many embodiments comprises a region proposal network 116 to propose regions of interest, which then undergo region of interest pooling 118 to highlight regions of interest within image data and generate weighting parameters. The second stage 108 of many embodiments involves pixel-to-pixel prediction based on weighting parameters. A number of embodiments obtain the weighting parameters from first stage 106. In second stage 108, resultant data 120 from backbone 104 is upsampled and combined via element-wise summing with data 122 arising from one or more pooling layers 114.

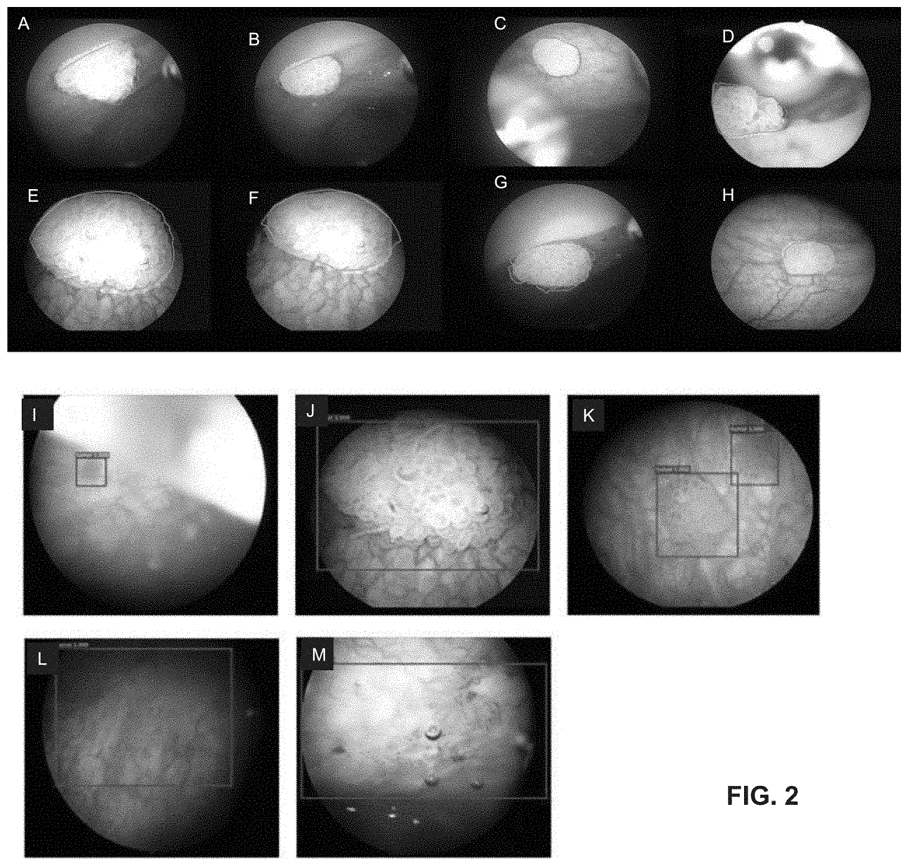

The resulting use of convolutional neural networks 110 within many embodiments involves inputting image data (e.g., video) 124 into the backbone 104. A first stage 106 of many embodiments highlights regions of interest 126 into the image data, while a second stage 108 performs image segmentation 128 on the image data. FIGS. 2A-2H illustrate tumor segmentation of certain embodiments, while FIGS. 2I-2M illustrate the highlighting of regions of interest in accordance with many embodiments.

Certain algorithms used in embodiments will be augmented to cope with a variety of challenges. For example, some embodiments implement heuristic weighting to features identified within cystoscopic imaging. Utilizing heuristic weighting will allow for the algorithm to cope with data imbalance, when the amount of normal tissue is in greater abundance than abnormal tissue (e.g., tumors). In such embodiments, the heuristic weighting renormalizes the data to improve sensitivity and/or specificity.

Further embodiments will also augment inter- and intraclass distances. For example, these embodiments will make distances between features from the similar pathologies closer to each other, while increasing distances between features from different pathologies. By augmenting inter- and intraclass distances, these embodiments will improve sensitivity and/or specificity in the embodiments.

### Training Machine Learning Models

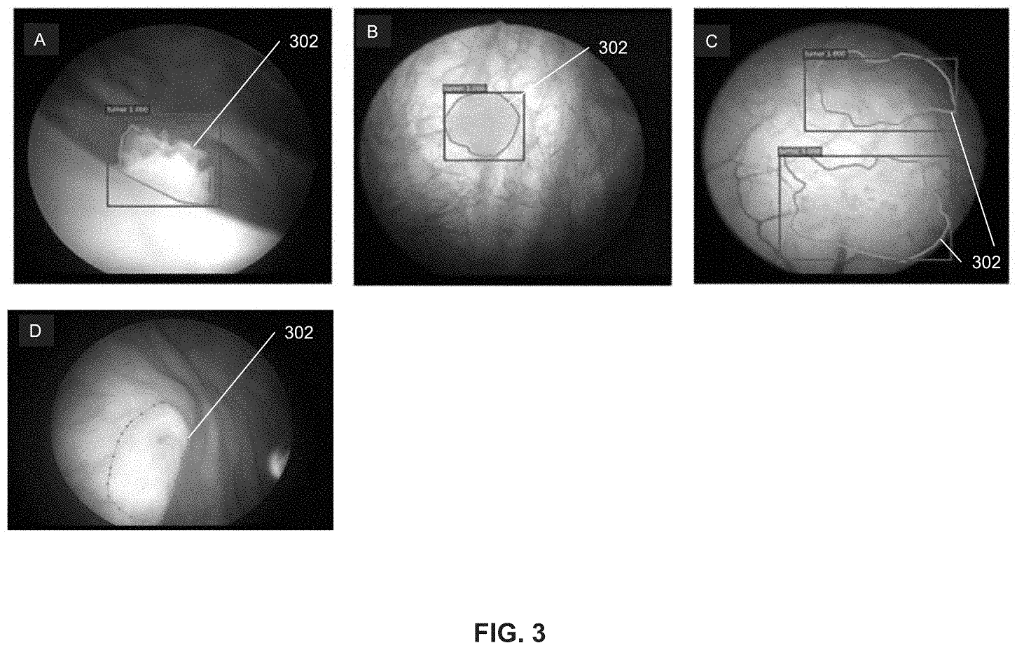

Many embodiments will train the machine learning algorithm by using supervised or semi-supervised learning. Certain embodiments will train an algorithm using videos from cystoscopic exams that have certain tissues annotated for abnormal tissues. Abnormal tissues include papillary tumors, flat bladder tumors, inflammatory lesions, and cystitis. Turning to FIGS. 3A-3C, annotated tumors are identified by boundaries 302. Further embodiments also train the algorithm with normal or benign physiologies and artifacts, such as ureteral orifices, bladder neck, air bubbles, and other benign features, such as illustrated in FIG. 3D.

Many embodiments will train using white light cystoscopy, while additional embodiments will use blue light cystoscopy videos, and more embodiments will use videos from both white-and blue-light cystoscopy. Integrating blue-light cystoscopy data can help facilitate the annotation process of diagnostically challenging flat tumors and cancers. Further embodiments will include details in the training data for cancer grade, stage, histology and resection margin status.

Further embodiments will include patient information in training the algorithm to improve detection or decisions regarding certain features identified within an individual. Relevant patient information can include age, sex/gender, medical history, and any information that may be relevant for diagnosis. Medical history can include underlying health concerns, such as obesity, diabetes, cancer history, prior issues with urinary tract (e.g., infections and inflammation), prior issues with the bladder (e.g., infections and inflammation), and/or other information that is relevant for bladder health. Additionally, information about prior bladder tumors, including location, size, and resection, can be input with the training data.

Methods for Diagnosing Cancer with AI-Enabled Cystoscopy

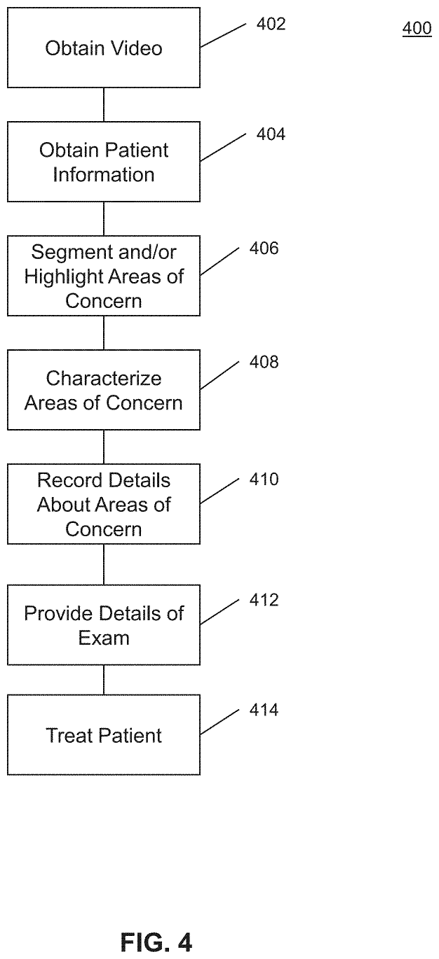

Turning to FIG. 4, a method 400 for diagnosing bladder cancer using AI-enabled Cystoscopy is illustrated. At 402, many embodiments obtain video from a live (e.g., ongoing) cystoscopic exam. In some embodiments, the video is obtained as a live feed from an ongoing cystoscopic exam, while certain embodiments will obtain video from a pre-recorded cystoscopic exam. For pre-recorded videos, the videos can be saved locally or remotely such as on a local hard drive, flash drive, server, or other storage device capable of storing video data.

Certain embodiments will obtain information about a patient from which the video is obtained at 404. These embodiments will obtain such information as age, sex/gender, medical history, and any other information that may be relevant for diagnosis. Medical history can include underlying health concerns, such as obesity, diabetes, cancer history, prior issues with urinary tract (e.g., infections and inflammation), prior issues with the bladder (e.g., infections and inflammation), and/or other information that is relevant for bladder health. Additionally, information about prior bladder tumors, including location, size, and resection, can be obtained at 404.

At 406, numerous embodiments will segment and/or highlight areas of concern, including abnormal tissue (e.g., tumors, lesions, and/or other areas of concern), present in the video. Segmentation and/or highlighting in many embodiments will use one or more machine learning algorithms, such as those described herein. In several embodiments using live imagery, highlighting of abnormal tissues will be placed on the video screen or other viewing device of a practitioner performing the cystoscopic exam. In these embodiments, live or real-time highlighting of areas of concern will guide the practitioner to obtain more images of the area of concern, including additional angles, close-ups, and/or any view that can aid in identifying, classifying, and/or characterizing the area of concern.

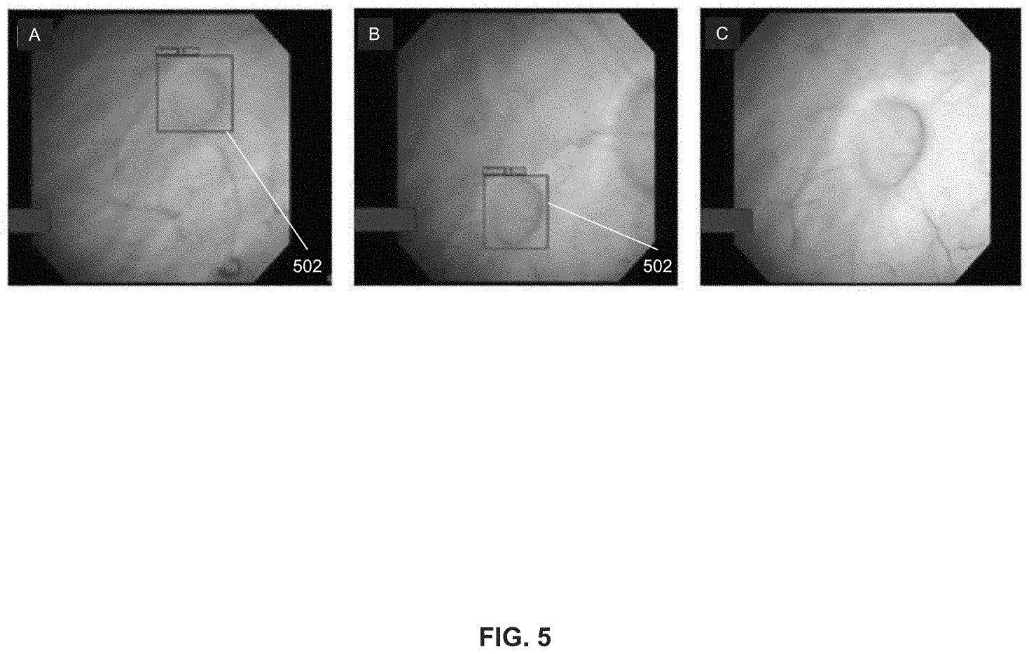

Additional embodiments will characterize the area of concern at 408. The characterization process can include identifying the area of concern (e.g., inflammation, a tumor, or benign tissue). Further embodiments will locate the area of concern and/or determine the size of an area of concern. If an area of concern is a tumor, certain embodiments determine type of tumor (e.g., flat or papillary). Some embodiments will further characterize tumors for cancer grade, stage, histology, and resection margin. Additional embodiments determine whether an area is benign based on patient information. For example, an area of inflammation may be considered benign in one patient (e.g., 32-year old woman with no history of bladder issues) but remain flagged as an area of concern in others (e.g., 70-year old man with a history of bladder cancer). If the area of concern is benign, such as a benign phenomenon or normal physiological feature (e.g., ureteral orifice, bladder neck, etc.), certain embodiments will exclude the area of concern (e.g., remove highlighting from a video monitor). An example of the removal of highlighting is illustrated in FIGS. 5A-5C, where FIGS. 5A-5B highlight 502 a phenomenon, which upon closer inspection (FIG. 5C) is determined to be benign, thus removing the highlighting and excluding the benign feature from further processing.

Further embodiments will record information determined about abnormal tissue at 410. The recordation process includes storing details about abnormal tissue to media, such as hard drives, servers, etc. for future use. Such details can include locations, sizes, types of abnormal tissue, number of tumors, and other relevant information for the patient undergoing the cystoscopic exam. Further embodiments will record metadata about the exam, including date of analysis, type of cystoscopy (e.g., white light, blue light, etc.), patient information (e.g., patient identifiers), and/or any other relevant information.

At 412, numerous embodiments will provide details of the cystoscopic examination to a practitioner. The details provided to a practitioner can be tabulated summaries of the details determined within this method, including locations, sizes, etc. further embodiments provide representative images of the areas of concern. In certain embodiments the details are provided as overlays or guidance to a practitioner during a follow-up cystoscopic exam, such as a more intensive exam or post-resection exam. For example, some embodiments will help a practitioner to identify whether the area of concern is improving, growing, etc. during a follow-up cystoscopic exam. Additionally, embodiments can allow the practitioner to identify whether the region has been completely resected or needs an additional resection. Some embodiments that provide live overlays of video will alert a practitioner to areas of concern that are no longer identified to be of concern—for example, if a prior cystoscopic exam revealed 9 tumors, but a subsequent exam only reveals 8 tumors, an alert can be provided to the practitioner to reexamine areas that were not identified during the subsequent exam to assure full inspection of the bladder during the subsequent cystoscopic exam.

Further embodiments treat the patient at 414. Treatment of the patient can include resecting the tumor, introducing an anti-cancer drug to the bladder, introducing an anti-cancer drug to the tumor, or any other applicable treatment for bladder cancer, bladder tumor, or other abnormal tissue. Certain embodiments will treat the patient using guidance provided to a practitioner, such as described in 412, thus guiding a practitioner to one or more tumors or other abnormal tissue.

It should be noted that method 400 is illustrative of features that may be included in various embodiments. As such, certain embodiments will omit certain features, complete features in a different order than illustrate, or even combine certain features into a single unit. For example, certain embodiments may combine segmenting and/or highlighting areas of concern 406, characterizing areas of concern 408, and recording details 410 into one or two features, rather than as three individual features. Further embodiments may also omit treatment, where resection, introducing anti-cancer drugs, or another treatment is not necessary following a subsequent cystoscopic exam. And, additional embodiments will repeat certain features, such that the segmenting and/or highlighting 406 can be repeated multiple times for purposes including to refine the segmentation and/or highlighting of certain features.

Further embodiments include non-transitory machine-readable media, where the media contains instructions that when read by a processor direct the processor to accomplish one or more of the features described in method 400. Additionally, certain embodiments are systems comprising

### Performance of Many Embodiments

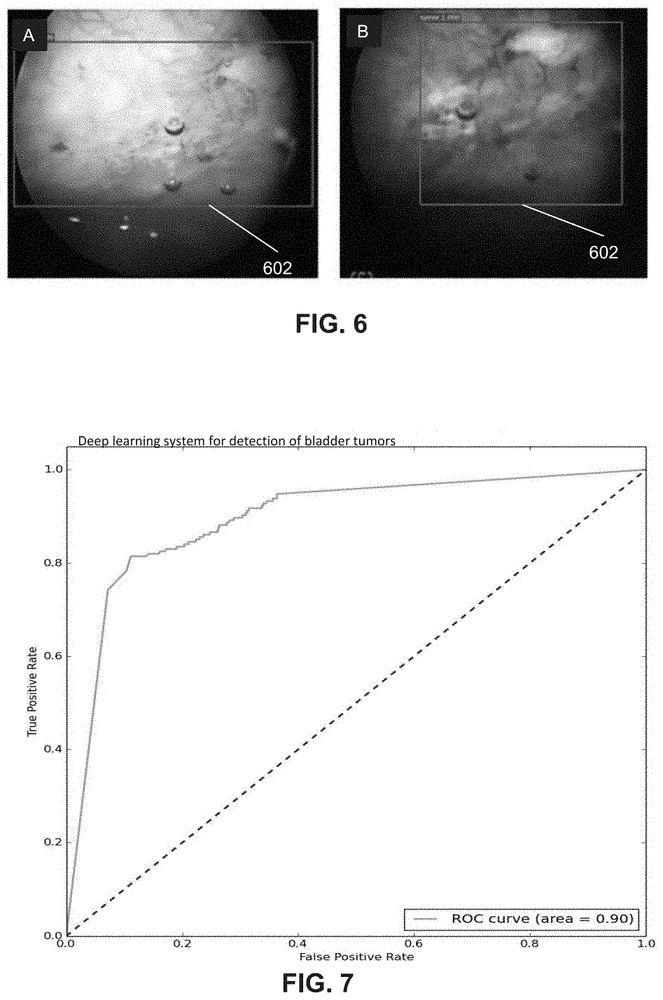

Turning to FIGS. 6A-6B, many embodiments are capable of performing as well as blue light cystoscopy without the need of extra equipment or procedures, such as an investment in blue light systems or injection or introduction of the dyes used in blue light cystoscopy. In FIG. 6A, a flat lesion is identified by highlighting 602 in an embodiment using white light cystoscopy. The flat lesion was confirmed via blue light cystoscopy, as highlighted 602 in FIG. 6B. Additionally, Table 1 illustrates performance evaluation from an embodiment. The algorithm was constructed using a training group (n=95 subjects) from the initial development dataset and tested in 5 subjects for initial performance evaluation. There were 130 cancers in the development dataset (43 low grade, stage Ta; 61 high grade, stage Ta; 17 high grade, stage T1; 9 high grade, stage T2). All video frames were reviewed and 611 frames containing histologically confirmed papillary urothelial carcinoma were labeled. Additionally, FIG. 7 illustrates an area under the curve of the receiver operating characteristic curve of an embodiment that is determined on a training set across a range of thresholds. FIG. 7 illustrates one curve that was selected to achieve optimal sensitivity and specificity.

### Exemplary Embodiments

Although the following embodiments provide details on certain embodiments of the inventions, it should be understood that these are only exemplary in nature, and are not intended to limit the scope of the invention.

### Example 1

### Machine Learning

Methods: Videos of office-based cystoscopy or transurethral resection of bladder tumors performed at the Veterans Affairs Palo Alto Health Care System (VAPAHCS) between 2016 and 2019 were obtained from patients undergoing evaluation for, or treatment of, bladder cancer. Patients with tumors found on cystoscopy subsequently underwent TURBT, and videos of biopsied lesions were correlated to final histopathology. Cystoscopies with no abnormalities identified were classified as benign. Informed consent was obtained from all participants and the study protocol was approved by the Stanford University Institutional Review Board and VAPAHCS Research and Development Committee. With IRB approval, videos of office-based cystoscopy and transurethral resection of bladder tumor from 100 subjects were prospectively collected and annotated. For algorithm development, video frames (n=611) containing histologically confirmed papillary bladder cancer were selected and tumor outlined (green line, Figure). Bladder neck, ureteral orifices, and air bubbles were labeled for exclusion learning. This embodiment used an image analysis platform based on convolutional neural networks, was developed to evaluate videos in two stages: 1) recognition of frames containing abnormal areas and 2) segmentation of regions within the frame occupied by tumor. A training set was constructed based on 95 subjects (417 cancer and 2,335 normal frames). A validation set was constructed based on 5 subjects (211 cancer, 1,002 normal frames).

Results: In the validation set, per-frame sensitivity was 88% (186/211) and per-tumor sensitivity was 90% (9/10) with a per-frame specificity of 99% (992/1002).

Conclusion: We have created a deep-learning algorithm that accurately detects papillary bladder cancers. Computer augmented cystoscopy may aid in diagnostic decision-making to improve diagnostic yield and standardize performance across providers.

### Example 2

### Algorithm Development

Methods: For algorithm development, video frames (n=611) containing histologically confirmed papillary bladder cancer were selected and tumors outlined using LabelMe annotating software. (See Russell BC, Torralba A, Murphy KP, Freeman WT. LabelMe: a database and web-based tool for image annotation. 2008 http://people.csail.mit.edu/brussell/research/AIM-2005-025-new.pdf (accessed Mar. 12, 2019); the disclosure of which is hereby incorporated by reference in its entirety.) Flat lesions were excluded from the development dataset as their margins could not be accurately delineated. Bladder neck, ureteral orifices, and air bubbles were labeled for exclusion learning. This embodiment an image analysis platform based on convolutional neural networks, was developed to evaluate videos in two stages: 1) recognition of frames containing abnormal areas and 2) segmentation of regions within the frame occupied by tumor with subsequent generation of a target box over the area of interest. A training set of 95 subjects (417 cancer and 2,335 normal frames) and a validation set of 5 subjects (211 cancer, 1,002 normal frames) were constructed. The area under the curve of the receiver operating characteristic curve of this embodiment was determined on the training set across a range of thresholds, and one was selected to achieve optimal sensitivity and specificity (FIG. 7). Per-frame and per-tumor sensitivity and per-frame specificity of the validation cohort of the development dataset were calculated.

Conclusion: We have created a deep-learning algorithm that accurately detects papillary bladder cancers. Computer augmented cystoscopy may aid in diagnostic decision-making to improve diagnostic yield and standardize performance across providers.

### Example 3

### Algorithm Testing

Methods: After initial validation, the algorithm threshold for detection was locked and the system evaluated prospectively. Fifty-four patients (57 videos) were recruited to evaluate the performance of this embodiment in detecting bladder cancer. There were no exclusion criteria and all patients undergoing cystoscopy or TURBT at the VAPAHCS between November 2018 and March 2019 were eligible. Videos obtained of cystoscopy and TURBT were analyzed using the present algorithm. Patient demographics, final histopathology, and video specifications were obtained (Table 1). Sensitivity for tumor detection was determined on a per-frame and per-tumor basis. Specificity was determined on a per-frame basis using videos from the benign cystoscopy cohort. Pearson's chi-square test was done to compare the proportion of frames marked inappropriately as cancerous within the benign cohort to the accurately identified cancerous frames within the tumor cohort.

Conclusion: We have created a deep-learning algorithm that accurately detects papillary bladder cancers. Computer augmented cystoscopy may aid in diagnostic decision-making to improve diagnostic yield and standardize performance across providers.

### Example 4

### Algorithm Testing

Methods: In many embodiments, a deep-learning algorithm for the detection of bladder tumors was developed using 141 videos from 100 patients undergoing TURBT for suspected bladder cancer. The training set contained 2,335 normal frames and 417 labeled frames containing histologically confirmed bladder tumors. The prospective cohort consisted of 57 videos from 54 patients. Of these, 34 (59.6%) were in-office flexible cystoscopy videos and 23 (40.4%) were TURBTs.

Results: In the validation subset, the per-frame sensitivity for tumor detection was 88% (95% CI, 83.0-92.2%) and 90% of tumors were accurately identified. The specificity was 99% (95% CI, 98.2%-99.5%).

Cystoscopy was normal in 31 videos, and a total of 44 tumors (42 papillary, 2 flat) were identified in the remaining 26 videos. A total of 20,643 frames were generated from the benign cystoscopies and 284 frames were falsely identified as malignant. A total of 38,872 frames were generated from tumor-containing cystoscopies and 6857 of 7542 tumor-containing frames were identified as malignant. Per-frame sensitivity and specificity were 90.9% (95% CI, 90.3%-91.6%) and 98.6% (95% CI, 98.5%-98.8%), respectively. Per-tumor sensitivity was 95.5% (95% CI, 84.5%-99.4%).

A mean of 665 frames were generated per benign cystoscopy and 1231 per tumor-identifying cystoscopy. In a normal cystoscopy, an average of 9.2 frames were incorrectly identified as abnormal using this embodiment whereas in a tumor-identifying cystoscopy an average of 155.8 frames-per-tumor were detected by the algorithm. Significantly more frames were identified by the algorithm in the tumor-identifying cystoscopies as compared to benign (12.7% vs 1.4%; p<0.001).

Conclusion: Feasibility of using this embodiment real-time was demonstrated with a frames-per-second processing speed allowing for real time or near real time use.

Numerous embodiments incorporate a deep-learning algorithm that accurately detects papillary bladder cancers. Additionally, many embodiments utilize a computer augmented cystoscopy may aid in diagnostic decision-making to improve diagnostic yield and standardize performance across providers.

### Example 5

### AI-Enabled Cystoscopy

Background: Deep learning applications of endoscopy, particularly in real time clinical settings, pose challenges that are distinct from static image interpretation of radiological and histological images. Challenges for bladder tumor identification include: 1) the low contrast between pathological lesions and surrounding area, 2) irregular and fuzzy lesion borders, 3) varying imaging light conditions, and 4) class or data imbalance (where the training data are usually skewed toward the nonpathological images).

Methods: To address the challenges with bladder tumor and cancer detection, the network architecture was enhanced by integrating two additional constraints, unbalanced discriminant (UD) loss and category sensitivity (CS) loss, to facilitate the extraction of discriminative image features (60). The UD loss aims to reduce the classification error caused by the imbalance of training datasets in the numbers of pathological and normal images. The CS loss is introduced based on the intuition that, if images Xᵢ and Xⱼ belong the same category, the corresponding features fᵢ and fⱼ calculated after the fully connected layer of the network should be close in the learned feature space. Otherwise, the fᵢ and fⱼ should be separated from each other. CS loss helps to minimize the intra-class variations of the learned features while maintaining the inter-class distances within the batch.

Results: With the joint supervision of UD loss and CS loss, a more robust deep learning model was trained. The experimental results achieved polyp detection accuracy of 93.19%, showing that the model can characterize accurately the endoscopic images. A detailed comparison of this embodiment with five existing methods was carried out and the results showed that our model outperforms the existing approaches (Table 2), as measured by using the assessment metrics of accuracy, recall, precision, F1, and FPR, where F1 and FPR measure a test's accuracy and false positive rate, respectively. Calculations of F1 and FPR are detailed in Equations 1 and 2, below:

$$\begin{matrix}F 1 = F 1 = \frac{2 * precision * recall}{precision + recall} & ( Eq . 1 ) \\ FPR = \frac{{N}_{FP}}{{N}_{Fp} + {N}_{TN}} & ( Eq . 2 )\end{matrix}$$

Table 2 illustrates results of a comparison of other models compared to this embodiment. Baseline methods 4 and 5 illustrate inferior performance by using only a single loss constraint (UD or CS) in learning deep features.

Conclusion: Feasibility of using this embodiment real-time was demonstrated with a frames-per-second processing speed allowing for real time or near real time use. Numerous embodiments incorporate a deep-learning algorithm that accurately detects papillary bladder cancers. Additionally, many embodiments utilize a computer augmented cystoscopy may aid in diagnostic decision-making to improve diagnostic yield and standardize performance across providers.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

TABLE 1
Patient demographics and tumor characteristics for development
and prospective data sets.

| | Development Datataset | | Validation Dataset | |
|---|---|---|---|---|
| | Training | Test | Normal | Tumor |
| Data Acquisition | 2016 - 2018 | | 2018 - 2019 | |
| Source | TURBT | TURBT | Clinic | Clinic + |
| | | | | TURBT |
| Patients | 95 | 5 | 31 | 23 |
| Videos | 136 | 5 | 31 | 26 |
| Nomal Frames | 2,335 | 1,002 | 20,643 | 31,330 |
| Tumor Frames | 417 | 211 | — | 7542 |
| Tumor number | 120 | 10 | — | 44 |
| Histology | | | | |
| Inverted papilloma | 0 | 0 | | 1 |
| LG Ta | 42 | 1 | | 13 |
| CIS | 0 | 0 | | 3 |
| HG Ta | 54 | 7 | | 15 |
| HG T1 | 15 | 2 | | 9 |
| HG T2 | 9 | 0 | | 3 |
| True Positives | — | 186 | — | 6,857 |
| False Negatives | — | 25 | — | 685 |
| True Negatives | — | 992 | 20,359 | 23,382 |
| False Positives | — | 10 | 284 | 406 |
| Per-Frame Sensitivity, | — | 88.2 | — | 90.9 |
| % (95% CI) | | (83.0-92.2) | | (90.3-91.6) |
| Per-Tumor Sensitivity, | — | — | — | 95.5 |
| % (95% CI) | | | | (84.5-99.4) |
| Per-Frame Specificity, | — | 99.0 | — | 98.6 |
| % (95% CI) | | (98.2-99.5) | | (98.5-98.8) |

LG, low grade;
HG, high grade;
CIS, carcinoma in situ;
CI, confidence interval.
True positives were defined as histologically confirmed bladder cancers marked with a
CystoNet alert;
False negatives were histologically confirmed bladder cancers without a CystoNet alert;
True negatives were frames containing normal bladder mucosa (either biopsy proven benign or deemed normal by the practicing urologist and not biopsied) with no alert;
False positives were normal bladder mucosa with an alert.
Per-tumor sensitivity is defined as algorithm sensitivity for detection of a histologically confirmed bladder cancer in at least one frame.

TABLE 2
Example of embodiment performance as compared to other models

| | Accuracy | Recall | Precision | F1 | FPR |
|---|---|---|---|---|---|
| | (%) | (%) | (%) | (%) | (%) |
| Baseline 1 (VGG-16) | 84.97 | 56.92 | 59.17 | 58.02 | 7.86 |
| Baseline 2 | 85.72 | 57.86 | 61.28 | 59.37 | 7.24 |
| (ResNet-50) | | | | | |
| Baseline 3 | 87.52 | 59.11 | 63.39 | 61.18 | 6.81 |
| (DenseNet) | | | | | |
| Baseline 4 | 90.28 | 75.85 | 68.94 | 72.23 | 6.75 |
| (DenseNet-UD) | | | | | |
| Baseline 5 | 90.97 | 82.56 | 69.23 | 75.31 | 7.31 |
| (DenseNet-CS) | | | | | |
| This Embodiment | 93.19 | 90.21 | 74.51 | 81.83 | 5.93 |

1. A method for identifying a bladder tumor comprising:
obtaining a video of a cystoscopic exam;
segmenting an area of concern present in the video;
recording details about the area of concern; and
providing details about the area of concern to a practitioner.

2. The method of claim 1, wherein the obtaining step is obtained from a live cystoscopic exam.

3. The method of claim 2, wherein the live cystoscopic exam is accomplished using white light cystoscopy.

4. The method of claim 1, wherein the segmenting an area of concern step uses a machine learning algorithm comprising a convolutional neural network.

5. The method of claim 4, wherein the convolutional neural network is trained with annotated cystoscopic video.

6. The method of claim 5, wherein the annotated cystoscopic video includes annotations of abnormal tissues and benign physiologies.

7. The method of claim 4, wherein the convolutional neural network comprises two stages.

8. The method of claim 4, wherein the convolutional neural network has a first stage and a second stage, wherein the first stage highlights an area of concern and the second stage segments a tumor.

9. The method of claim 1, wherein the providing step is accomplished via video overlay during a subsequent cystoscopic exam.

10. The method of claim 1, further comprising obtaining patient information, wherein the patient information comprises at least one of the group consisting of: age, sex, gender, and medical history.

11. The method of claim 1, wherein the segmenting step highlights the area of concern on a video monitor.

12. The method of claim 1, further comprising characterizing the area of concern.

13. The method of claim 12, wherein the characterizing step comprises at least one of the group consisting of: identifying the area of concern, locating the area of concern, and determining the size of the area of concern.

14. The method of claim 12 wherein the characterizing step comprises identifying the area of concern and excluding the area of concern, if the area of concern is benign.

15. The method of claim 1, further comprising treating the patient for a tumor.

16. The method of claim 15, wherein treating the patient comprises at least one of the group consisting of: resecting the tumor, introducing an anti-cancer drug to the bladder, and introducing an anti-cancer drug to the tumor.

17. A method for treating a bladder tumor comprising:
obtaining a video from a live white light cystoscopic exam;
obtaining patient information, wherein the patient information comprises at least one of the group consisting of: age, sex, gender, and medical history;
segmenting an area of concern present in the video using a machine learning algorithm comprising a convolutional neural network trained with annotated cystoscopic video, wherein the annotated video includes annotations of abnormal tissues and benign physiologies, wherein the segmenting step highlights the area of concern on a video monitor;
characterizing the area of concern, wherein characterizing the area of concern comprises at least one of the group consisting of: identifying the area of concern, locating the area of concern, and determining the size of the area of concern, wherein characterizing the area of concern further comprises excluding the area of concern, if the area of concern is benign;
recording details about the area of concern;
providing details about the area of concern to a practitioner; and
treating the patient for a tumor; wherein treating the patient comprises at least one of the group consisting of: resecting the tumor, introducing an anti-cancer drug to the bladder, and introducing an anti-cancer drug to the tumor.
